(19) United States
(12) Patent Application Publication
(10) Pub. No.: US 2021/0338613 A1
(43) Pub. Date: Nov. 4, 2021

(54) FORMULATIONS AND METHODS FOR TRANSDERMAL ADMINISTRATION OF KETONES

(71) Applicant: DYVE BIOSCIENCES, INC., Thousand Oaks, CA (US)

(72) Inventors: Ryan Beal, Thousand Oaks, CA (US); Luke Gonzales, Thousand Oaks, CA (US); Kilmar Martinez, Thousand Oaks, CA (US); Brandon Sand, Thousand Oaks, CA (US); Lisa Misell, Thousand Oaks, CA (US); Nathan Fitzsimmons, Thousand Oaks, CA (US)

(73) Assignee: DYVE BIOSCIENCES, INC., Thousand Oaks, CA (US)

(21) Appl. No.: 17/282,990

(22) PCT Filed: Oct. 6, 2019
(86) PCT No.: PCT/US2019/054908

§ 371 Date: Apr. 5, 2021

Related U.S. Application Data
(60) Provisional application No. 62/742,172, filed on Oct. 5, 2018.

(51) Int. Cl. A61K 31/19; A61K 9/00; A61K 47/24; A61P 3/10; A61P 25/28; A61P 35/00; A61P 3/04

(52) U.S. Cl. CPC A61K 31/19; A61K 9/0014; A61K 47/24; A61P 3/04; A61P 25/28; A61P 35/00; A61P 3/10

(57) ABSTRACT

A formulation for transdermal delivery of one or more ketone components through the skin of a subject, comprising: a ketone component in an amount between about 10-60% w/w; a penetrant portion in an amount less than about 60% w/w, and water in an amount less than about 50% w/w.

### CROSS-REFERENCE TO RELATED APPLICATIONS

This application is related to and claims priority to U.S. Provisional Application Ser. No. 62/742,172 filed Oct. 5, 2018, all incorporated by reference in their entirety herein.

### BACKGROUND

The subject of this patent application relates generally to formulations and methods for transdermal administration of ketones.

Inducing ketosis can be very beneficial to metabolic processes, but it comes with significant barriers. Ketosis is the body's natural response to a prolonged period of energy deficit (starvation ketosis), as a result of very low carbohydrate (CHO) and very high fat intake (nutritional ketosis aka “keto diet”), or as part of the clinical manifestation of diseases such as uncontrolled diabetes. In these cases, the body breaks down fat and produces ketone bodies in order to meet energy requirements. Ketone bodies are especially useful because they are a viable (and 70% more effective) alternative to glucose for brain cell energy needs. However, caloric or CHO restriction present issues of their own.

Nutritional ketosis induced by the ketogenic diet (KD) has been shown to have a number of therapeutic applications. For example, the KD has been used as an effective non-pharmacological therapy for pediatric intractable seizures since the 1920s. In addition to epilepsy, the ketogenic diet has elicited significant therapeutic effects f or weight loss and type-2 diabetes. Several studies have shown significant weight loss on a high fat, low carbohydrate diet without significant elevations of serum cholesterol. Another study demonstrated the safety and benefits of long-term application of the KD in T2D patients. Patients exhibited significant weight loss, reduction of blood glucose, and improvement of lipid markers after eating a well-formulated KD for 56 weeks. Recently, researchers have begun to investigate the use of the KD as a treatment for acne, polycystic ovary syndrome (PCOS), cancer, amyotrophic lateral sclerosis (ALS), traumatic brain injury (TBI) and Alzheimer's disease (AD) with promising preliminary results.

Recently it has been shown that ketosis can be generated by introducing exogenous ketone bodies via oral supplementation. This has been tested orally via transesterifying ethyl (R)-3-hydroxybutyrate with (R)-1,3-butanediol using lipase (Cox et al., 2016). This provides a way to study human ketone metabolism independent of caloric or CHO deficit. It was found that ketone body oxidation has a regulatory role, controlling the preferential use and release of other substrates, such as fat and glucose.

Applicant hereby incorporate herein by reference any and all patents and published patent applications cited or referred to in this application. Aspects of the present disclosure fulfill these needs and provide further related advantages as described in the following summary.

### SUMMARY

Aspects of the present disclosure teach certain benefits in construction and use which give rise to the exemplary advantages described below.

The present disclosure solves the problems described above by providing formulations and methods of transdermal delivery of ketones.

In one aspect, disclosed herein are formulations for transdermal delivery of one or more ketone components through the skin of a subject, comprising: a ketone component in an amount between about 10-60% w/w; a penetrant portion in an amount less than about 60% w/w, and water in an amount less than about 50% w/w.

In another aspect disclosed herein is a method of inducing ketosis to treat a disorder and/or treating a disorder with ketone supplementation in a subject, wherein the method comprises administering an effective amount of a formulations for transdermal delivery of one or more ketone components through the skin of a subject, comprising: a ketone component in an amount between about 10-60% w/w; a penetrant portion in an amount less than about 60% w/w, and water in an amount less than about 50% w/w.

Other features and advantages of aspects of the present disclosure will become apparent from the following more detailed description, taken in conjunction with the accompanying drawings, which illustrate, by way of example, the principles of aspects of the disclosure.

### DETAILED DESCRIPTION

In one aspect, disclosed herein is a formulation for transdermal delivery ketone components through the skin of a subject, comprising: a ketone component in an amount between about 10-60% w/w; a penetrant portion in an amount less than about 60% w/w, and water in an amount less than about 50% w/w.

In another aspect disclosed herein is a method of inducing ketosis to treat a disorder and/or treating a disorder with ketone supplementation in a subject, wherein the method comprises administering an effective amount of a formulations for transdermal delivery of one or more ketone components through the skin of a subject, comprising: a ketone component in an amount between about 10-60% w/w; a penetrant portion in an amount less than about 60% w/w, and water in an amount less than about 50% w/w.

Nutritionally induced ketosis has been shown to have numerous therapeutic and performance benefits. Exogenous ketone supplementation has been shown to induce ketosis and deliver the therapeutic and performance benefits without the challenges of severe dietary restriction and modification. Current means of exogenous ketone delivery are limited to oral supplementation, with ketone ester supplementation showing the most favorable results. Oral supplementation, however, is not ideal as absorption and elimination kinetics are complex, oral supplementation has been shown to decrease endogenous ketone supplementation, and currently available oral supplements are unpalatable (ketone esters) and/or present electrolyte imbalance and cation overload risks (ketone salts).

Topically delivered ketones circumvent these challenges and provide a patient-friendly. This disclosure provides for a method of safe and effective topical delivery of actives that induce ketosis. The disclosure embodies the actives current used in oral supplements today (primarily ketone esters) and pH ketone components to aid in bioavailability, synergistically improve efficacy, and protect against ketoacidosis. These elements can be combined in a single use formulation or alternatively in separately applied formulations.

### Therapeutic Benefits of Ketosis

When comparing athletes during an exercise time trial, those who ingested Ketone Bodies (KE) and CHO showed better results than those just on CHO. Specifically, in KE+CHO subjects, blood lactate was significantly decreased during and after exercise. The KE+CHO subjects also showed greater lipid oxidation (Intramuscular lipids fell by 24% during KE+CHO, but only 1% on CHO). In addition, KE+CHO subjects exhibited a modest increase in physical capacity during the time trial. Through the introduction of ketone bodies, the re-prioritization of fuel sources can greatly increase energy efficiency in the body, as well as increase human performance.

In addition, in oncology, ketone supplementation has been shown to decrease tumor cell viability and prolongs survival of mice with metastatic cancer. Cancer cells express an abnormal metabolism characterized by increased glucose consumption. Previous studies indicate that unlike healthy tissues, cancer cells are unable to effectively use ketone bodies for energy.

Widespread mitochondrial pathology has been observed in most if not all tumors examined, including decreased mitochondrial number, abnormal ultrastructural morphology, mitochondrial swelling, abnormal fusion-fission, partial or total cristolysis, mtDNA mutations, altered mitochondrial membrane potential and abnormal mitochondrial enzyme presence or function, among others. As ketone bodies are metabolized exclusively within the mitochondria, cancer cells with impaired mitochondrial function are unable to efficiently metabolize ketone bodies for energy.

Ketone bodies possess many characteristics that can impair cancer cell survival and proliferation.

a. Ketone bodies inhibit glycolysis, thus decreasing the main pathway of energy production for cancer cells.

b. Cancer cells thrive in an environment of elevated reactive oxygen species (ROS) production but are very sensitive to even small changes in redox status. Ketones decrease mitochondrial ROS production and enhance endogenous antioxidant defenses in normal cells, but not in cancer cells. Ketone metabolism in healthy cells near the tumor may inhibit cancer cell growth by creating a less favorable redox environment for their survival.

c. Ketone bodies are transported into the cell through the monocarboxylate transporters (MCTs), which are also responsible for lactate export. It has been shown that inhibiting MCT1 activity or inhibiting lactate export from the cell dramatically decreases cancer cell growth and survival. Ketones may impair cancer cells indirectly by competitive inhibition of the MCTs, decreasing critical lactate export from the cell.

d. Recently, it has been demonstrated that βHB acts as an endogenous HDAC inhibitor at millimolar concentrations easily achieved through fasting, CR or ketone supplementation such as with a ketone ester (KE). Thus, ketone bodies may elicit their anticancer effects by altering the expression of oncogenes and tumor suppressor genes under control of the cancer epigenome.

Clearly, ketone bodies exhibit several unique characteristics that support their use as a metabolic therapy for cancer. Ketone administration elicited anticancer effects in vitro and in vivo independent of glucose levels or calorie restriction.

The current disclosure delivers on the promise of exogenous ketone supplementation as a means to induce ketosis without the biochemical and practical challenges inherent in current delivery approaches. The ketone component refers to ketone esters, ketone salts, and related compounds such as acetoacetate, beta-hydroxybutyrate, or combinations thereof. In some embodiments, the ketone component an organic compound with the structure RC(═O)R′ or derivatives thereof, where R and R′ can be a variety of carbon-containing substituents.

Topical Ketone Esters or Ketone Salts: Avoidance of GI metabolic complexity and minimization of hepatic negative feedback loop on endogenous ketone production: Topically delivered KE and/or KS in formulations of the disclosure herein.

Topical KE and KS metabolites: Avoid need for carboxylesterase conversion of KE or KS to D-βHB and/or betahydroxybutyrate dehydrogenase conversion of D-βHB to acetoacetate: Topically delivered D-βHB and/or acetoacetate in formulations of the disclosure herein.

Prevention of electrolyte imbalance and cation overload: Biologically balance formulation of KS or use of KE and/or use of downstream metabolites, D-βHB and/or acetoacetate.

Maintenance of physiologic pH: Ketone esters lead to acetoacetic acid and beta-hydroxybutyric acid, both of which are acidic. If levels of these ketone bodies are too high, the pH of the blood drops, resulting in ketoacidosis, a complication of untreated Type I diabetes, and sometimes in end stage Type II: Topically delivered pH ketone components in formulations of the disclosure.

In addition, the formulations could be applied sequentially or alternatively at various times through the day.

The surprising effects achieved by the formulations and methods of the present discloser are attributable to an improved formulation that enhances delivery of a ketone components through the skin. In some embodiments, the formulation employs penetrants described US2009/0053290 ('290), W02014/209910 ('910), and WO2017/127834. The present formulations may include a nonionic surfactant. Applicant has found that by employing ketone components with particle sizes as disclosed herein, delivered with the penetrants as disclosed herein, and in some embodiments providing a combination of a nonionic surfactant and a polar gelling agent, the penetration capabilities of the ketone components of the resulting formulation and the effective level of delivery of the ketone components have been enhanced. This enhanced level of penetration was also achieved using significantly less lecithin than anticipated or none at all. This result was completely unexpected as it was believed that a somewhat higher concentration of lecithin organogel were responsible for the level of penetration achieved by prior art formulations.

Briefly, the penetrants described in the above-referenced US and PCT applications are based on combinations of synergistically acting components. Many such penetrants are based on combinations of an alcohol, such as benzyl alcohol to provide a concentration of 0.5-20% w/w of the final formulation with lecithin organogel present in the penetrant to provide 10-70% w/w of the formulation. These penetrants are also useful when the agent is a ketone component as disclosed herein, but less lecithin organogel may be required—e.g. less than 35% w/w when the ketone component is present at high concentration as disclosed herein.

The formulations of the disclosure may be prepared in a number of ways. Typically, the components of the formulation are simply mixed together in the required amounts. However, it is also desirable in some instances to, for example, carry out partial dissolution of a ketone component and then add a separate preparation containing the components aiding the delivery of the ketones in the form of a carrier. The concentrations of these components in the carrier, then, will be somewhat higher than the concentrations required in the final formulation. Thus, ketone component may first be partially dissolved in water and then added to a carrier comprising an alcohol, lecithin and optionally a combination of a nonionic surfactant and polar gelling agent, or of ionic detergent. Alternatively, some subset of these components can first be mixed and then “topped off” with the remaining components either simultaneously or sequentially. The precise manner of preparing the formulation will depend on the choice of ketone components and the percentages of the remaining components that are desirable with respect to that ketone component. In some embodiments, the water is less than about 85% w/w, 50% w/w, or 30% w/w of the formulation.

In some embodiments, the one or more ketone components are formulated with Aveeno® moisturizers, cream, oils, lotions; Jergens® moisturizers, cream, oils, lotions; Honest Company® moisturizers, cream, oils, lotions; Dermologica® moisturizers, cream, oils, lotions; or St. Ives™ moisturizers, cream, oils, lotions. In some embodiments, the commercial lotions, moisturizers, etc. are formulated with the ketone component in an amount between about 10-60% w/w.

The penetrant portion is a multi-component mixture, whereby the particular concentrations of the penetration enhancers are informed in part by the particle size of the ketone component. The formulation enables the ketone component to become bio-available to the target site within minutes of topical administration. In some embodiments, the penetrant portion comprises an alcohol in an amount less than 5% w/w of the formulation.

Subjects of the disclosure herein, in addition to humans, include veterinary subjects, wherein formulations suitable for these subjects are also appropriate. Such subjects include livestock and pets as well as sports animals such as horses and greyhounds.

One aspect of the invention is a method to inhibit cancer growth and metastasis, including diminution of cancer mass by non-systemic parenteral, including topical administration of ketone components as disclosed herein, including solid tumors and melanomas.

The formulations comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

For topical administration, and in particular transdermal administration, the formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290 ('290), W02014/209910 ('910), and WO2017/127834. In addition to formulations with penetrants, transdermal delivery can be effected by mechanically disrupting the surface of the skin to encourage penetration, or simply by supplying the formulation applied to the skin under an occlusive patch.

Alternatively, the penetrant portion comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Lecithin organogel is a combination of lecithin with a gelling component. Suitable gelling components also include isopropyl palmitate, ethyl laurate, ethyl myristate and isopropyl myristate. In some embodiments, the formulation comprises a gelling agent in an amount less than 5% w/w of the formulation. Certain hydrocarbons, such as cyclopentane, cyclooctane, trans-decalin, trans-pinane, n-pentane, n-hexane, n-hexadecane may also be used. Thus, an important permeation agent is a lecithin organogel, wherein the combination resulting from lecithin and the organic solvent acts as a permeation agent. In some embodiments, the formulation comprises less than about 7% w/w, less than about 12% w/w, less than about 30% w/w lecithin, or less than about 50% w/w lecithin. In some embodiments, the penetrant portion comprises lecithin organogel, an alcohol, a surfactant, and a polar solvent. In some embodiments, the lecithin organogel is a combination of soy lecithin and isopropyl palmitate. In some embodiments, the penetrant portion comprises lecithin and isopropyl palmitate, undecane, isododecane, isopropyl stearate, or a combination thereof. In some embodiments, the formulation comprises Lipmax™ in an amount between about 1-50 w/w or an equivalent 50/50 mixture of isopropyl palmitate and lecithin. Lecithin organogels are not always clear or thermodynamically stable, but are viscoelastic, and biocompatible phases composed of phospholipids and appropriate organic liquid. An example of a suitable lecithin organogel is lecithin isopropyl palmitate, which is formed when isopropyl palmitate is used to dissolve lecithin. The ratio of lecithin to isopropyl palmitate may be 50:50. Illustrated below in the Examples is a formulation containing soy lecithin in combination with isopropyl palmitate; however, other lecithins could also be used such as egg lecithin or synthetic lecithins. Various esters of long chain fatty acids may also be included. Methods for making such lecithin organogels are well known in the art. In most embodiments, the lecithin organogel is present in the final formulation is less than about 20% w/w. In those compositions used to alleviate pain or in anhydrous compositions, the concentration of lecithin organogel may be as low as 0.5% w/w, 1% w/w, 10% w/w, 20% w/w or 50% w/w. In some embodiments, the penetrant portion comprises a mixture of xanthan gum, lecithin, sclerotium gum, pullulan, or a combination thereof in an amount less than 2% w/w, 5% w/w, or 10% w/w of the formulation. In some embodiments, the formulation comprises Siligel™ in an amount between about 1-5% w/w or 5-15% w/w, or an equivalent mixture of xanthan gum, lecithin, sclerotium gum, and pullulan. In some embodiments, the penetrant portion comprises a mixture of caprylic triglycerides and capric triglycerides in amount less than 2% w/w, 8% w/w, or 10% w/w of the formulation. In some embodiments, the formulation comprises Myritol® 312 in an amount between about 0.5-10% w/w, or an equivalent mixture of caprylic triglycerides and capric triglycerides.

In some embodiments, the penetrant portion is in an amount between about 10-90% w/w or 10-50% w/w of the formulation. In some embodiments, the penetrant portion comprises phosphatidyl choline in amount less than 7% w/w, less than 12% w/w, or 18% w/w of the formulation. In some embodiments, the penetrant portion comprises a phospholipid in amount less than 20% w/w or 50% w/w of the formulation. In some embodiments, the penetrant portion comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the penetrant portion comprises cetyl alcohol in amount less than 2% w/w, 5% w/w, or 8% w/w of the formulation. In some embodiments, the formulation comprises benzyl alcohol in an amount less than about 2% w/w, 5% w/w, or 8% w/w. In some embodiments, the penetrant portion comprises stearic acid in an amount less than 2% w/w, 5% w/w, or 8% w/w of the formulation. In some embodiments, the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 30% w/w or in amount less than 12% w/w of the formulation.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semi-solid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

The lecithin organogels are formulated with an additional component to assist in the formation of micelles or vascular structures. In one approach, the organogels are formulated with a polar component such as water, glycerol, ethyleneglycol or formamide, in particular with water. In general, a nonionic detergent such as a poloxamer in aqueous solution is used to top off. Certain detergents, such as Tween® 80 or Span® 80 may be used as alternatives. The percentage of these components in the anhydrous forms of the composition is in the range of 1-15% w/w. In these essentially anhydrous forms, powdered or micronized nonionic detergent is used to top off, typically in amounts of 1-30% w/w of the formulation.

An additional component in the formulations of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are incl tided. Due to the aromatic group present in a permeation enhancement formulation such as benzyl alcohol, the molecule has a polar end (the alcohol end) and a non-polar end (the benzene end). This enables the agent to dissolve a wider variety of formulation components. The alcohol concentration is substantially lower than the concentration of the lecithin organogel in the composition.

In some embodiments, as noted above, the performance of the formulations is further improved by including a nonionic detergent and polar gelling agent or including a powdered surfactant. In both aqueous and anhydrous forms of the composition, detergents, typically nonionic detergents are added. In general, the nonionic detergent should be present in an amount between about 1% w/w to 30% w/w of the formulation. Typically, in the compositions wherein the formulation is topped off with a polar or aqueous solution containing detergent, the amount of detergent is relatively low—e.g., 2-25% w/w, or 5-15% w/w or 7-12% w/w of the formulation. However, in compositions that are essentially anhydrous and are topped-off by powdered detergent, relatively higher percentages are usually used—e.g., 20%-60% w/w.

In some embodiments, the penetrant portion further comprises a detergent portion in an amount between about 1 to 70% w/w or 1-60% w/w of the formulation. In some embodiments, the nonionic detergent provides suitable handling properties whereby the formulations are gel-like or creams at room temperature. To exert this effect, the detergent, typically a poloxamer, is present in an amount between about 2-12% w/w of the formulation, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, the formulation also contains surfactant, typically, nonionic surfactant at 2-25% w/w of the formulation along with a polar solvent wherein the polar solvent is present in an amount at least in molar excess of the nonionic surfactant. In these embodiments, typically, the composition comprises the above-referenced amounts of lecithin organogel and benzyl alcohol along with a ketone component with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® FI27, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 1-30% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that may also be included in the formulations are fatty acids, terpenes, lipids, and cationic, and anionic detergents. In some embodiments, the formulation further comprises tranexamic acid in an amount less than 2% w/w, 5 w/w, or 10% w/w of the formulation. In some embodiments, the formulation further comprises a polar solvent in an amount less than 2% w/w, 5% w/w, 10% w/w, or 20 w/w of the formulation. In some embodiments, the formulation further comprises a humectant, an emulsifier, an emollient, or a combination thereof. In some embodiments, the formulation further comprises almond oil in an amount less than about 5% w/w. In some embodiments, the formulation further comprises a mixture of thermoplastic polyurethane and polycarbonate in an amount less than about 5% w/w. In some embodiments, the formulation further comprises phosphatidylethanolamine in an amount less than about 5% w/w. In some embodiments, the formulation further comprises an inositol phosphatide in an amount less than about 5% w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-l-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxpropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and a-lipoic acid typically in concentrations of 0.1%-2.5% w/w.

In some applications, it is desirable to adjust the pH of the formulation to assist in permeation or to adjust the nature of the ketone component and/or of the target compounds in the subject. In some instances, the pH is adjusted to a level of pH 9-11 or 10-11 which can be done by providing appropriate buffers or simply adjusting the pH with base.

In some applications, in particular when the formulation includes an anesthetic, epinephrine or an alternate vasoconstrictor, such as phenylephrine or epinephrine sulfate may be included in the formulation if a stabilizing agent is present. Otherwise, the epinephrine should be administered in tandem since epinephrine is not stable at alkali pH.

In any of the anesthetic compositions, it may be desirable to administer the epinephrine in tandem with the transdermal anesthetic. Alternatively, treatment of the epinephrine with a chelator, such as the iron chelator Desferal® may stabilize the epinephrine sufficiently to include it in the transdermal formulation.

Another active agent is Withaferin A. Withaferin A inhibits tumor metastasis and manifests other anti-cancer activities, e.g., inhibition of the neovascularzation associated with carcinoma, as well as cell proliferation. Withaferin A is also a leptin sensitizer with strong anti-diabetic properties that could induce healthy weight loss and beneficial effects on glucose metabolism.

Other agents include anti-metastatic agents including inhibitors of the src homology region 2-containing protein tyrosinase phosphatase (Sh p2). A multiplicity of inhibitors of this activity is known, including Fumosorine, PHPS (NSC-87877) and NSC-117199, phenylhydrazonopyrazolone sulfonate (PHPS1), DCA, cryptotanshinone, 11-B08 and #220-324, metalloproteinases-2 and -9 (MMP-2 and MMP-9) and certain cathepsins, in particular B, D and L.

Other agents include inhibitors of E-cadherin and of epidermal growth factor receptor (EGFR). Known inhibitors include erlotinib, an anti-integrin drug (Cilengitide), Cariporide, Eniporide and Amiloride.

The formulations may include other components that act as excipients or serve purposes other than active anti-tumor effects. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft® (which is a mixture of thermoplastic polyurethane and polycarbonate). Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of the formulation itself. The application of the formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying the formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

A wide variety of therapeutic agents may be used in the formulations, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply effect transport across the skin into a localized subdermal location, such as treatment of nail fungus or modulation of hair growth or may effect systemic delivery such as is desirable in some instances where vaccines are used.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with the formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the ketone component to pass through the epidermis to the dermis at least, and, if systemic administration is achieved, through the dermis itself.

For administration of anesthetics as the therapeutic agent, the local anesthetic may be one or more of the following: benzocaine, lidocaine, tetracaine, bupivacaine, cocaine, etidocaine, mepivacaine, pramoxine, prilocaine, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. Combinations of anesthetic agents may also be used. The anesthetic agent{s) are included in the composition in effective amount(s). Depending on the anesthetic(s) the amounts of anesthetic or combination is typically in the range of 1% w/w to 50% w/w. The compositions of the invention provide rapid, penetrating relief that is long lasting. The pain to be treated can be either traumatic pain and/or inflammatory pain.

In one embodiment, the anesthetic is administered to relieve the pain associated with invasive fat deposit removal. Specific removal of fat deposits has been attractive for both health and cosmetic reasons. Among the methods employed are liposuction and injection of a cytolytic agent for fat such as deoxycholic acid (DCA). For example, a series of patents issued or licensed to Kythera Biopharmaceuticals is directed to methods and compositions for non-surgical removal of localized fat that involves injecting compositions containing DCA or a salt thereof. Representative issued patents are directed to formulation (U.S. Pat. NO. 8,367,649); method-of-use (U.S. Pat. Nos. 8,846,066; 7,622,130; 7,754,230; 8,298,556); and synthetic DCA (U.S. Pat. No. 7,902,387).

In this aspect of the invention, conventional invasive fat removal techniques are employed along with administering a pain-relieving effective agent—typically lidocaine or related anesthetics via transdermal administration. In some embodiments, the pain-relieving transdermal formulation is applied to the area experiencing pain immediately before, during or immediately after the invasive fat-removal procedure.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

In another embodiment, nutrients are supplied via transdermal administration. There are many occasions in which the formulations of the invention are useful. For athletes, the formulations can deliver to tired muscles sufficient amounts of a neutralizing agent for lactic acid, such as ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation especially in those areas of their musculature that are involved in performing workouts and, therefore, need to consume large numbers of calories. These nutrients can be supplied directly rather than requiring oral ingestion which is counterproductive and relatively slow.

Emergency medical treatment of individuals requiring, for example, blood balancing agents including electrolytes and readily-metabolized nutrients, such as glucose, that would otherwise be administered intravenously can instead be non-invasively treated by massaging the formulation through the skin and thus permitting systemic delivery so that levels in the bloodstream are altered.

In addition to these applications, it has been noted that the administration of nutrients according to the invention also assuages feelings of hunger. Therefore, the formulations of the invention and methods of the invention are useful in promoting weight loss as the caloric intake required to assuage feelings of hunger is lower than that ordinarily experienced by consuming food conventionally. Thus, in addition to individuals requiring extra calories or metabolic balancers because of exertion and in addition to those unable to feed themselves orally, suitable subjects for the methods of the invention include individuals seeking to control their caloric intake in order to adjust their weight. In view of the generally acknowledged obesity epidemic in the United States in particular, this is an important group of subjects benefitting from the methods of the invention.

It is clear that the nature of the desired. ingredients will vary depending on the object of the administration. Simple nutrients such as amino acids, glucose, fructose, simple fats, various vitamins, cofactors and antioxidants as well as somewhat more complex foodstuffs can be administered as well as neutralizing agents, depending on the need.

In some embodiments, the components for athletic performance include beta-alanine, L-carnitine, adenosine triphosphate, dextrose, creatine monohydrate, beta hydroxy-betamethylbutyrate (HMB), branched chain amino acids (leucine, isoleucine, valine), glutathione, sodium phosphate, and caffeine. Components for medical nutrition include amino acids, dextrose, lipids, Na⁺, K⁺, Ca²⁺, Mg²⁺, acetate, Cl⁻, P, multivitamin, and trace elements. While components for weight loss include conjugated linoleic acids, ephedra, caffeine, and salicin.

Certain embodiments of formulations provided herein may be supplemented with formulation components described in greater detail in the inventor's related applications, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

In one aspect, disclosed herein is a formulation for transdermal delivery ketone components through the skin of a subject, comprising: a ketone component in an amount between about 10-60% w/w; a penetrant portion in an amount less than about 60% w/w, and water in an amount less than about 50% w/w.

In some embodiments, the penetrant portion further comprises a detergent portion in an amount between about 1 to 30% w/w.

In some embodiments, the detergent portion comprises a nonionic surfactant in an amount between about 2-25% w/w of the penetrant portion; and a polar solvent in an amount less than 5% w/w of the penetrant portion.

In some embodiments, the ketone component is in an amount between about 20-60% w/w of the formulation.

In some embodiments, the penetrant portion is in an amount between about 10-60% w/w of the formulation.

In some embodiments, the water is in an amount between about 15-40% w/w of the penetrant portion of the formulation.

In some embodiments, the water is deionized water.

In some embodiments, the penetrant portion comprises an alcohol in an amount less than 10% w/w of the formulation.

In some embodiments, the penetrant portion comprises lecithin organogel, an alcohol, a surfactant, and a polar solvent.

In some embodiments, the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof in amount less than 30% w/w of the formulation.

In some embodiments, the ketone component comprises Sodium 3-hydroxybutyrate in amount less than 35% w/w of the formulation.

In some embodiments, the penetrant portion comprises cetyl alcohol in amount less than 5% w/w of the formulation.

In some embodiments, the penetrant portion comprises Almond Oil in an amount less than 5% w/w of the formulation.

In some embodiments, the penetrant portion comprises benzyl alcohol in an amount less than 5% w/w of the formulation.

In some embodiments, the penetrant portion comprises ethanol in an amount less than 5% w/w of the formulation.

In some embodiments, the penetrant portion comprises glycerine in an amount less than 5% w/w of the formulation.

In some embodiments, the penetrant portion comprises propylene glycol in an amount less than 8% w/w of the formulation.

In some embodiments, the formulation comprises a gelling agent in an amount less than 20% w/w of the formulation.

In some embodiments, the ketone component is a salt milled to a particle size less than 70 pm.

In some embodiments, the salt of the ketone component is solubilized in the formulation in an amount less than 10% w/w of the formulation.

In some embodiments, the formulation further comprises tranexamic acid in an amount less than 5% w/w of the formulation.

In some embodiments, the formulation further comprises a polar solvent in an amount less than 5% w/w of the formulation.

In some embodiments, the formulation further comprises a humectant, an emulsifier, an emollient, or a combination thereof.

In some embodiments, the formulation has a pH of 7-10.5.

In some embodiments, the formulation for transdermal delivery of a ketone component through the skin of a subject, comprises a formulation of Table 1, Table 2, Table 3, Table 4, Table 5, or Table 6.

In another aspect disclosed herein is a method of inducing ketosis to treat a disorder and/or treating a disorder with ketone supplementation in a subject, wherein the method comprises administering an effective amount of a formulations for transdermal delivery of one or more ketone components through the skin of a subject, comprising: a ketone component in an amount between about 10-60% w/w; a penetrant portion in an amount less than about 60% w/w, and water in an amount less than about 50% w/w.

In some embodiments, the disorder is pediatric intractable seizures.

In some embodiments, the disorder is epilepsy.

In some embodiments, the disorder is type-2 diabetes.

In some embodiments, the disorder is obesity.

In some embodiments, the method induces weight loss.

In some embodiments, the method reduces blood glucose levels.

In some embodiments, the disorder is acne.

In some embodiments, the disorder is polycystic ovary syndrome.’

In some embodiments, the disorder is cancer.

In some embodiments, the disorder is amyotrophic lateral sclerosis.

In some embodiments, the disorder is traumatic brain injury.

In some embodiments, the disorder is Alzheimer's disease.

In some embodiments, the disorder is metabolic syndrome, glycogen storage disease, autism, Parkinson's disease, Glucose transporter 1 (GLUT1) deficiency syndrome, PDH deficiency, PFK deficiency, McArdle disease, Multiple scleross, nonalcoholic fatty liver disease, migraines, depression, headaches, narcolepsy, or cardiac ischemia.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-25% w/w;

Water in an amount between about 10-35% w/w;

Sodium 3-hydroxybutyrate in an amount between about 10-60% w/w;

Propylene glycol in an amount between about 1-5% w/w;

Almond oil in an amount between about 1-6% w/w;

Glycerin in an amount between about 0.25-5% w/w;

Cetyl alcohol in an amount between about 0.5-5% w/w; and

Ethanol in an amount between about 0.5-3% w/w.

In some embodiments, the formulation comprises:

Lipmax™ in an amount between about 10-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 5-20% w/w;

Sodium 3-hydroxybutyrate in an amount between about 10-60% w/w;

Durosoft® in an amount between about 0.1-5% w/w;

Propylene glycol in an amount between about 1-5% w/w;

Cetyl alcohol in an amount between about 0.5-5% w/w, and

Ethanol in an amount between about 0.5-3% w/w.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-20% w/w;

Water in an amount between about 10-35% w/w;

Sodium 3-hydroxybutyrate in an amount between about 10-60% w/w;

Propylene glycol in an amount between about 1-8% w/w;

Almond oil in an amount between about 1-6% w/w;

Glycerin in an amount between about 0.25-5% w/w;

Cetyl alcohol in an amount between about 0.5-5% w/w; and

Ethanol in an amount between about 0.5-3% w/w.

In some embodiments, the formulation comprises:

Lipmax™ in an amount between about 10-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 10-25% w/w;

Sodium 3-hydroxybutyrate in an amount between about 10-60% w/w;

Durosoft® in an amount between about 0.1-5% w/w;

Ethanol in an amount between about 0.5-3% w/w.

Propylene glycol in an amount between about 1-5% w/w; and

Cetyl alcohol in an amount between about 0.5-5% w/w.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-25% w/w;

Water in an amount between about 10-35% w/w;

Sodium 3-hydroxybutyrate in an amount between about 10-60% w/w;

Glycerin in an amount between about 0.25-3% w/w;

Propylene glycol in an amount between about 1-8% w/w;

Almond oil in an amount between about 1-6% w/w;

Cetyl alcohol in an amount between about 0.5-5% w/w; and

Ethanol in an amount between about 0.5-3% w/w.

In some embodiments, the formulation comprises:

Lipmax™ in an amount between about 15-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 5-25% w/w;

Sodium 3-hydroxybutyrate in an amount between about 10-60% w/w;

Propylene glycol in an amount between about 1-5% w/w;

Cetyl alcohol in an amount between about 0.5-5% w/w;

Durosoft® in an amount between about 0.1-5% w/w; and

Ethanol in an amount between about 0.5-3% w/w.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-25% w/w;

Water in an amount between about 10-35% w/w;

Sodium acetoacetate in an amount between about 10-60% w/w;

Propylene glycol in an amount between about 1-5% w/w;

Almond oil in an amount between about 1-6% w/w;

Glycerin in an amount between about 0.25-5% w/w;

Cetyl alcohol in an amount between about 0.5-5% w/w; and

Ethanol in an amount between about 0.5-3% w/w.

In some embodiments, the formulation comprises:

Lipmax™ in an amount between about 10-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 5-20% w/w;

Sodium acetoacetate in an amount between about 10-60% w/w;

Durosoft® in an amount between about 0.1-5% w/w;

Propylene glycol in an amount between about 1-5% w/w;

Cetyl alcohol in an amount between about 0.5-5% w/w, and

Ethanol in an amount between about 0.5-3% w/w.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-20% w/w;

Water in an amount between about 10-35% w/w;

Sodium acetoacetate in an amount between about 10-60% w/w;

Propylene glycol in an amount between about 1-8% w/w;

Almond oil in an amount between about 1-6% w/w;

Glycerin in an amount between about 0.25-5% w/w;

Cetyl alcohol in an amount between about 0.5-5% w/w; and

Ethanol in an amount between about 0.5-3% w/w.

In some embodiments, the formulation comprises:

Lipmax™ in an amount between about 10-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 10-25% w/w;

Sodium acetoacetate in an amount between about 10-60% w/w;

Durosoft® in an amount between about 0.1-5% w/w;

Ethanol in an amount between about 0.5-3% w/w.

Propylene glycol in an amount between about 1-5% w/w; and

Cetyl alcohol in an amount between about 0.5-5% w/w.

In some embodiments, the formulation comprises:

Lipmax™ in an amount between about 5-20% w/w;

Benzyl alcohol in an amount between about 0.5-3% w/w;

Menthol in an amount between about 0.1-2% w/w;

Pluronic® in an amount between about 5-25% w/w;

Water in an amount between about 10-35% w/w;

Sodium acetoacetate in an amount between about 10-60% w/w;

Glycerin in an amount between about 0.25-3% w/w;

Propylene glycol in an amount between about 1-8% w/w;

Almond oil in an amount between about 1-6% w/w;

Cetyl alcohol in an amount between about 0.5-5% w/w; and

Ethanol in an amount between about 0.5-3% w/w.

In some embodiments, the formulation comprises:

Lipmax™ in an amount between about 15-35% w/w;

Benzyl alcohol in an amount between about 0.5-3% w/w;

Menthol in an amount between about 0.1-2% w/w;

Pluronic® in an amount between about 10-30% w/w;

Deionized water in an amount between about 5-25% w/w;

Sodium acetoacetate in an amount between about 10-60% w/w;

Propylene glycol in an amount between about 1-5% w/w;

Cetyl alcohol in an amount between about 0.5-5% w/w;

Durosoft® in an amount between about 0.1-5% w/w; and

Ethanol in an amount between about 0.5-3% w/w.

In applying the formulations of the invention, the formulation itself is simply placed on the skin and spread across the surface and/or massaged to aid in penetration. The amount of formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of the formulation to the skin and prevents distortion of the formulation by evaporation in some cases.

The schedule of application is dependent on the nature of the treatment being administered. Repeated application is often desirable, for example, during intermittent types of exercise. Alternatively, the formulation may be left in place, preferably covered during athletic performance. Application to supply nutrients to patients may also be for prolonged periods of time.

The composition may be applied to the skin using standard procedures for application such as a brush, a syringe, a gauze pad, a dropper, or any convenient applicator. More complex application methods, including the use of delivery devices, may also be used, but are not required.

In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

It has surprisingly been found that using the formulations and methods of the present invention, nutrients can be supplied in effective amounts transdermally either preferentially to a desired area or systemically. Other agents which may be helpful in maintaining appropriate metabolic balance, for example, in muscles, can also be successfully administered in this manner. Thus, the need for oral administration, intravenous or other invasive administration of ketones is obviated.

As is apparent from the discussion above, the penetrants of the invention have wide application and are applicable to a number of drug delivery scenarios and can be adapted to the administration of a wide variety of therapeutic agents in addition to ketones. The extent of delivery is dependent on the application—simple transdermal transmission to a site of action as in the case of local anesthetics, treatment of fingernails or toenails, or volume and texture enhancement of tissue are examples of local delivery. On the other hand, delivery of nutrients and in some cases antiviral agents and anti-infective agents as well as cannabinoids and pain killers such as NSAIDs can be systemic.

Notably, local anesthetics can readily be delivered using the formulations of the invention by simple application to the skin. In this case, the use of epinephrine is beneficial as is the use of alkali pH—e.g., pH 8-10. Because epinephrine is not stable at high pH's, either it should be delivered separately in tandem with the delivery of the anesthetic itself in a composition of suitable pH, or it may be stabilized by adding an appropriate stabilizing agent such as Desferal® in the context of the anesthetic composition itself.

In some embodiments, the disclosure is directed to administering a local anesthetic to a subject transdermally and a formulation which contains an effective amount of anesthetic along with 25%-70% w/w or 30%-60% w/w or 30%-40% w/w of lecithin organogel typically wherein the lecithin organogel comprises soy lecithin in combination with isopropyl palmitate or isopropyl myristate and benzyl alcohol in the range of 0.5%-20% w/w or 0.9%-2% w/w benzyl alcohol optionally including 1%-5% w/w or 2%-4% w/w menthol wherein the composition is topped off with a polar solution, typically an aqueous solution comprising 15%-50% w/w or 20%-40% w/w or 20%-30% w/w poloxamer, typically Pluronic® or alternatively may be an anhydrous composition comprising bile salts such as deoxycholic acid or sodium deoxycholate in the range of 4%-8% w/w, typically 6% w/w and the remainder of the composition powdered nonionic detergent, typically Pluronic®. As is known, bile salts are facial amphiphiles and include salts of taurocholic acid, glycocholic acid, taurochenodeoxycholic acid, glycochenodeoxycholic acid, cholic acid, deoxycholic acid Detergents are also useful in lieu of bile salts and include Tween® 80 and Span® 80. The pH of the compositions is adjusted to 9-11, typically 10-11. The formulations are applied to the desired area of the skin and may be covered, for example, with Saran™ wrap for a suitable amount of time. Following the treatment, the skin can be repaired by applying a composition comprising linoleic acid.

Similar formulations as described above are used wherein the active component is a nutrient or combination of nutrients, or a dicarboxylic anhydride. Systemic administration of nutrients is especially important as is the treatment of viral infection, bacterial infection or other microbial infection using standard methods. For smoking cessation, the therapeutic agent is cytisine, also known as baptitoxine and sophorine, and is an alcohol that occurs naturally in several plant genera.

Suitable therapeutic agents to be delivered in using the ketone formulations for treatment of post procedural bruising include helenalin, a sesquiterpene, a lactone as well as Vitamin K. The formulation based on helenalin may be accompanied by irradiation with light of wavelength 577-595 nm. Other therapeutic agents include Botox®, flavonoids, skin lighteners and materials that promote collagen biosynthesis.

### EXAMPLES

The following non-limiting examples are provided for illustrative purposes only in order to facilitate a more complete understanding of representative embodiments now contemplated. These examples are intended to be a mere subset of all possible contexts in which the formulations and methods may be utilized. Thus, these examples should not be construed to limit any of the embodiments described in the present specification, including those pertaining to formulations for transdermal delivery of ketones and/or methods and uses thereof. Ultimately, the formulations and methods may be utilized in virtually any context where transdermal delivery of ketones are desired.

### Example 1: Use of Topically Delivered Ketones to Increase Ketonemia

In this experiment, β-hydroxybutyrate (βHB) formulations of the disclosure were tested for its ability to increase ketonemia and compared to an orally delivered β-hydroxybutyrate (βHB) mineral salt mixture.

In vivo tests were performed as follows: Wistar rats were treated with either topically applied βHB or orally delivered βHB without any other dietary changes. The experiment was terminated after 4 weeks. Trial design was built to assess these primary outcomes: the effects on ketonemia, and blood lipid profile. Treatment groups randomized as follows:

a. Control Group

b. Treatment Group 1: 4.2% concentration βHB mineral salts solution in drinking water ad libitum

c. Treatment Group 2: 100 μL of βHB formulation applied topically twice daily. Formulations detailed below

TABLE 1
β-Hydroxybutyrate Formulation A

| Chemical Components | Wt % |
|---|---|
| Menthol | 0.50% |
| Cetyl Alcohol | 2.00% |
| Almond Oil | 3.00% |
| Lipmax | 16.00% |
| Benzyl Alcohol | 1.00% |
| Water | 24.50% |
| 30% Pluronic Gel | 15.00% |
| Glycerine | 0.50% |
| Propylene Glycol | 4.00% |
| Ethanol | 1.50% |
| Sodium 3-hydroxybutyrate | 32.00% |
| Total | 100.00% |

Both Treatment Groups showed significant increases in ketonemia. Treatment Group 1 (oral βHB) showed a mean increase of 22% in ketonemia during experimental evening (ZT 14-16), and a mean increase of 51% during experimental morning (ZT 0-1) vs Control Group. Treatment Group 2 (topical βHB) showed a mean increase of 28% in ketonemia during experimental evening (ZT 14-16), and a mean increase of 60% during experimental morning (ZT 0-1) versus Control Group.

After 4 weeks of treatment, both Treatment Groups showed significant improvement in lipid profile. Treatment Group 1 (oral βHB) showed a 49% reduction in LDL/HDL cholesterol ratio versus Control Group. Treatment Group 2 (topical βHB) showed a 55% reduction in LDL/HDL cholesterol ratio versus Control Group.

### Example 2: Use of Topically Delivered Ketones to Prolong Survival of Mice with Metastatic Cancer

In this experiment, β-hydroxybutyrate (βHB) in formulations of the disclosure was tested for its ability to decrease tumor cell viability and prolong survival and compared to an orally delivered diet supplemented with either 1,3-butanediol (BD) or a ketone ester (KE), which are metabolized to the ketone bodies βHB and acetoacetate.

In vivo tests were performed as follows: Adult male inbred VM mice were implanted subcutaneously with firefly luciferase-tagged syngeneic VM-M3 cells. The mice were then fed a standard diet with no topical treatment, a standard diet supplemented with either 1,3-butanediol (BD) or a ketone ester (KE), or a standard diet with topically applied βHB. Trial design was built to assess this primary outcome: survival time. Treatment groups randomized as follows:

a. Control Group: standard diet rodent chow

b. Treatment Group 1: standard diet rodent chow mixed with 20% 1,3-butanediol (BD) by volume and 1% saccharin for palatability

c. Treatment Group 2: standard diet rodent chow mixed with 10% ketone esters (KE) by volume and 1% saccharin for palatability

d. Treatment Group 3: standard diet rodent chow with 100 μL of βHB formulation applied topically twice daily. Formulations detailed below

TABLE 2
β-Hydroxybutyrate Formulation B

| Chemical Components | Wt % |
|---|---|
| Menthol | 0.50% |
| Cetyl Alcohol | 2.00% |
| Benzyl Alcohol | 1.00% |
| Lipmax | 25.00% |
| Deionized Water | 15.00% |
| Ethanol | 1.50% |
| Propylene Glycol | 2.00% |
| 30% Pluronic Gel | 20.00% |
| Sodium 3-hydroxybutyrate | 32.00% |
| Durosoft PK-SG | 1.00% |
| Total | 100.00% |

All Treatment Groups showed significant increases in survival time. Treatment Group 1 (oral BD) showed a mean increase of 50.6% in mean survival time versus Control Group (47.0 days vs 31.2 days). Treatment Group 2 (oral KE) showed a mean increase of 69.2% in mean survival time versus Control Group (52.8 days vs 31.2 days). Treatment Group 3 (topical βHB) showed a mean increase of 60.6% in mean survival time versus Control Group (50.1 days vs 31.2 days).

### Example 3: Use of Topically Delivered Ketones to Enhance Efficacy of PI3K inhibitors in Oncology

In this experiment, β-hydroxybutyrate (βHB) in formulations of the disclosure was tested for its ability to enhance the efficacy of PI3K inhibitors and compared to a ketogenic diet.

In vivo tests were performed as follows: C57/BL6 mice 8 weeks of age They were injected with 0.5-1×10% cells in a 1:1 mix of growth media and matrigel. and tumors were allowed to grow to a minimum diameter of 0.6 cm before the initiation of treatment. All mice were treated daily with the P3K inhibitor BKM120 (37.5 mg/kg) by oral gavage and were given either a normal chow diet, a ketogenic diet, or a normal chow diet with there topically applied βHB. Trial design was built to assess this primary outcome: survival time. Treatment groups randomized as follows:

a. Control Group

b. Treatment Group 1: BKM120 treatment with standard rodent chow

c. Treatment Group 2: BKM120 treatment with ketogenic rodent chow

d. Treatment Group 3: BKM120 treatment with standard rodent chow with 100 μL of βHB formulation applied topically twice daily. Formulations detailed below

TABLE 3
β-Hydroxybutyrate Formulation C

| Chemical Components | Wt % |
|---|---|
| Menthol | 0.50% |
| Cetyl Alcohol | 2.00% |
| Almond Oil | 3.00% |
| Lipmax | 16.00% |
| Benzyl Alcohol | 1.00% |
| Water | 24.50% |
| 30% Pluronic Gel | 15.00% |
| Glycerine | 0.50% |
| Propylene Glycol | 4.00% |
| Ethanol | 1.50% |
| Sodium 3-hydroxybutyrate | 32.00% |
| Total | 100.00% |

Treatment Groups 2 and 3 showed significant increases in survival time with all mice still alive after 30-day termination of experiment. Control group mice had a mean survival time of 6.1 days. Treatment Group 1 was not statistically different from Control at 6.7 days mean survival time.

### Example 4: Use of Topically Delivered Ketones to Enhance Physical and Cognitive Performance

In this experiment, β-hydroxybutyrate (βHB) in formulations of the disclosure was tested for its ability to enhance physical and cognitive performance and compared to a ketone ester diet: chow that is supplemented with (R)-3-hydroxybutyl (R)-3-hydroxybutyrate as 30% of calories.

In vivo tests were performed as follows: Wistar rats were treated with either topically applied βHB or a ketone ester diet. The experiment lasted for 5 days. Trial design was built to assess these primary outcomes: the effects on distance run on a treadmill, and on time to complete an 8 arm radial maze. Treatment groups randomized as follows:

a. Control Group: standard diet (calorically matched to all treatment groups)

b. Treatment Group 1: ketone ester diet with chow that is supplemented with (R)-3-hydroxybutyl (R)-3-hydroxybutyrate as 30% of calories

c. Treatment Group 2: standard diet (calorically matched to all treatment groups) plus 100 μL of βHB formulation applied topically twice daily. Formulations detailed below

TABLE 4
β-Hydroxybutyrate Formulation D

| Chemical Components | Wt % |
|---|---|
| Menthol | 0.50% |
| Cetyl Alcohol | 2.00% |
| Benzyl Alcohol | 1.00% |
| Lipmax | 25.00% |
| Deionized Water | 15.00% |
| Ethanol | 1.50% |
| Propylene Glycol | 2.00% |
| 30% Pluronic Gel | 20.00% |
| Sodium 3-hydroxybutyrate | 32.00% |
| Durosoft PK-SG | 1.00% |
| Total | 100.00% |

Both Treatment Groups showed significant increases in physical performance. Treatment Group 1 (ketone ester diet) showed a mean increase of 32% in treadmill distance vs control. Treatment Group 2 (topical βHB) showed a mean increase of 27% in treadmill distance vs control.

Both Treatment Groups showed significant increases in cognitive performance. Treatment Group 1 (ketone ester diet) showed a mean decrease of 38% in time to solve the maze vs control. Treatment Group 2 (topical βHB) showed a mean decrease of 34% in time to solve the maze vs control.

### Example 5: Use of Topically Delivered Ketones to Effect Blood Ketone and Glucose Levels

In this experiment, β-hydroxybutyrate (βHB) in formulations of the disclosure was tested for its ability to impact blood ketone and glucose levels and compared to oral delivery of various exogenous ketone supplements.

In vivo tests were performed as follows: Sprague-Dawley rats were treated with either topically applied βHB or given daily 10 g/kg dose via intragastric gavage of one of several oral supplements: 1,3-butanediol (BD), a sodium/potassium β-hydroxybutyrate (βHB) mineral salt (BMS), medium chain triglyceride oil (MCT), BMS+MCT 1:1 mixture, or 1,3 butanediol acetoacetate diester (KE). The experiment lasted for 28 days. Trial design was built to assess these primary outcomes: the effects on blood ketones and blood glucose. Blood samples were taken for analysis of glucose and βHB at baseline and, 0.5, 1, 4, 8, and 12 h post-treatment. Treatment groups randomized as follows:

a. Control Group

b. Treatment Group 1: daily 10 g/kg dose via intragastric gavage of 1,3-butanediol (BD)

c. Treatment Group 2: daily 10 g/kg dose via intragastric gavage of sodium/potassium β-hydroxybutyrate (βHB) mineral salt (BMS)

d. Treatment Group 3: daily 10 g/kg dose via intragastric gavage of medium chain triglyceride oil (MCT)

e. Treatment Group 4: daily 10 g/kg dose via intragastric gavage of BMS+MCT 1:1 mixture

f. Treatment Group 5: daily 10 g/kg dose via intragastric gavage of 1,3 butanediol acetoacetate diester (KE)

g. Treatment Group 6: 100 μL of βHB formulation applied topically daily. Formulations detailed below

TABLE 5
β-Hydroxybutyrate Formulation E

| Chemical Components | Wt % |
|---|---|
| Menthol | 0.50% |
| Cetyl Alcohol | 2.00% |
| Almond Oil | 3.00% |
| Lipmax | 16.00% |
| Benzyl Alcohol | 1.00% |
| Water | 24.50% |
| 30% Pluronic Gel | 15.00% |
| Glycerine | 0.50% |
| Propylene Glycol | 4.00% |
| Ethanol | 1.50% |
| Sodium 3-hydroxybutyrate | 32.00% |
| Total | 100.00% |

BMS +MCT (Treatment Group 4) (10 g/kg) and MCT (Treatment Group 3) (10 g/kg) elevated blood βHB levels within 30 min and remained significantly elevated for up to 12 h. BMS+MCT peaked at 8 h instead of at 4 h and MCT at 4 h instead of at 1 h. Blood βHB levels in the BMS group (Treatment Group 2) did not show significant elevation at any time point. Synthetically derived ketogenic supplements (KE and BD—Treatment groups 5 and 1) rapidly elevated blood βHB within 30 min and was sustained for 8 h. Treatment Group 6—Topical βHB, showed elevated blood βHB levels within 30 min and remained significantly elevated for up to 12 h.

MCT (10 g/kg), BMS+MCT (10 g/kg), and Topical βHB decreased blood glucose within 30 min and lasted through the 12 h time point. Rats supplemented with BMS had lower blood glucose compared to control at 12 h. Administration of BD and KE did not significantly change blood glucose levels at any time point during the 4-week study.

### Example 6: Use of Topically Delivered Ketones to Effect Blood βHB in Humans

In this experiment, β-hydroxybutyrate (βHB) in formulations of the disclosure was tested for its ability to impact blood ketone βHB concentrations and compared to oral delivery of various exogenous ketone supplements.

In vivo tests were performed as follows: healthy human participants were treated with either topically applied βHB or given ketone ester (KE) or ketone salt (KS) drinks. Blood was sampled hourly for 4 hours post treatment. Treatment groups randomized as follows:

a. Control Group

b. Treatment Group 1: ingestion of ˜24 g of βHB as a ketone ester (KE); (R)-3-hydroxybutyl (R)-3-hydroxybutyrate

c. Treatment Group 2: ingestion of ˜24 g of βHB as ketone salts (KS); sodium plus potassium βHB

d. Treatment Group 3: Single topical application of 20 g of βHB formulation. Formulations detailed below

TABLE 6
β-Hydroxybutyrate Formulation F

| Chemical Components | Wt % |
|---|---|
| Menthol | 0.50% |
| Cetyl Alcohol | 2.00% |
| Benzyl Alcohol | 1.00% |
| Lipmax | 25.00% |
| Deionized Water | 15.00% |
| Ethanol | 1.50% |
| Propylene Glycol | 2.00% |
| 30% Pluronic Gel | 20.00% |
| Sodium 3-hydroxybutyrate | 32.00% |
| Durosoft PK-SG | 1.00% |
| Total | 100.00% |

Blood d-βHB concentrations rapidly increased to a maximum of 2.8±0.2 mM following the KE, to 1.0±0.1 mM following the KS drink, and 2.0±0.2 mM following the topical βHB. d-βHB Tmax was ˜2-fold longer following KS drinks and ˜4 fold longer following topical βHB when compared to KE drinks.

In closing, regarding the exemplary embodiments of the present disclosure as shown and described herein, it will be appreciated that a formulation for transdermal delivery of one or more ketone components is disclosed and configured for methods of delivery of to a subject in need. Because the principles of the disclosure may be practiced in a number of configurations beyond those shown and described, it is to be understood that the disclosure is not in any way limited by the exemplary embodiments, but is generally directed to a formulation for transdermal delivery of one or more ketone components and is able to take numerous forms to do so without departing from the spirit and scope of the disclosure.

Certain embodiments of the present disclosure are described herein, including the best mode known to the inventor(s) for carrying out the disclosure. Of course, variations on these described embodiments will become apparent to those of ordinary skill in the art upon reading the foregoing description. The inventor(s) expect skilled artisans to employ such variations as appropriate, and the inventor(s) intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure otherwise claimed. No language in the present specification should be construed as indicating any non-claimed element essential to the practice of the disclosure.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

While aspects of the disclosure have been described with reference to at least one exemplary embodiment, it is to be clearly understood by those skilled in the art that the disclosure is not limited thereto. Rather, the scope of the disclosure is to be interpreted only in conjunction with the appended claims and it is made clear, here, that the inventor(s) believe that the claimed subject matter is the disclosure.

What is claimed is:

1. A formulation for transdermal delivery of one or more ketone components through the skin of a subject, comprising:
a ketone component in an amount between about 10-60% w/w;
a penetrant portion in an amount less than about 60% w/w, and
water in an amount less than about 50% w/w.

2. The formulation of claim 1, wherein the penetrant portion further comprises a detergent portion in an amount between about 1 to 30% w/w.

3. The formulation of claim 2, wherein the detergent portion comprises a nonionic surfactant in an amount between about 2-25% w/w of the penetrant portion; and a polar solvent in an amount less than 5% w/w of the penetrant portion.

4. The formulation of claim 1, wherein the ketone component is in an amount between about 20-60% w/w of the formulation.

5. The formulation of claim 1, wherein the penetrant portion is in an amount between about 10-60% w/w of the formulation.

6. The formulation of claim 1, wherein the water is in an amount between about 15-40% w/w of the formulation.

7. The formulation of claim 6, wherein the water is deionized water.

8. The formulation of claim 8, wherein the penetrant portion comprises an alcohol in an amount less than 10% w/w of the formulation.

9. The formulation of claim 7, wherein the penetrant portion comprises lecithin organogel, an alcohol, a surfactant, and a polar solvent.

10. The formulation of claim 1, wherein the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof in amount less than 30% w/w of the formulation.

11. The formulation of claim 1, wherein the ketone component comprises Sodium 3-hydroxybutyrate and/or sodium acetoacetate in amount less than about 35% w/w of the formulation.

12. The formulation of claim 1, wherein the penetrant portion comprises cetyl alcohol in amount less than 5% w/w of the formulation.

13. The formulation of claim 1 wherein the penetrant portion comprises Almond Oil in an amount less than 5% w/w of the formulation.

14. The formulation of claim 1 wherein the penetrant portion comprises benzyl alcohol in an amount less than 5% w/w of the formulation.

15. The formulation of claim 1 wherein the penetrant portion comprises ethanol in an amount less than 5% w/w of the formulation.

16. The formulation of claim 1 wherein the penetrant portion comprises glycerine in an amount less than 5% w/w of the formulation.

17. The formulation of claim 1 wherein the penetrant portion comprises propylene glycol in an amount less than 8% w/w of the formulation.

18. The formulation of claim 1, wherein the formulation comprises a gelling agent in an amount less than 20% w/w of the formulation.

19. The formulation of claim 1, wherein the ketone component is a salt milled to a particle size less than 70 μm.

20. The formulation of claim 19, wherein the salt of the ketone component is solubilized in the formulation in an amount less than 10% w/w of the formulation.

21. The formulation of claim 1, further comprising tranexamic acid in an amount less than 5% w/w of the formulation.

22. The formulation of claim 1, further comprising a polar solvent in an amount less than 5% w/w of the formulation.

23. The formulation of claim 1, further comprises a humectant, an emulsifier, an emollient, or a combination thereof.

24. The formulation of claim 1, wherein the formulation has a pH of 7-10.5.

25. A formulation for transdermal delivery of a ketone component through the skin of a subject, comprising a formulation of Table 1, Table 2, Table 3, Table 4, Table 5, or Table 6.

26. A method of inducing ketosis to treat a disorder and/or treating a disorder with ketone supplementation in a subject, wherein the method comprises administering an effective amount of a formulation according to any one of claims 1-25.

27. The method according to claim 26, wherein the disorder is pediatric intractable seizures.

28. The method according to claim 26, wherein the disorder is epilepsy.

29. The method according to claim 26, wherein the disorder is type-2 diabetes.

30. The method according to claim 26, wherein the disorder is obesity.

31. The method according to claim 26, wherein the method induces weight loss.

32. The method according to claim 26, wherein the method reduces blood glucose levels.

33. The method according to claim 26, wherein the disorder is acne.

34. The method according to claim 26, wherein the disorder is polycystic ovary syndrome.

35. The method according to claim 26, wherein the disorder is cancer.

36. The method according to claim 26, wherein the disorder is amyotrophic lateral sclerosis.

37. The method according to claim 26, wherein the disorder is traumatic brain injury.

38. The method according to claim 26, wherein the disorder is Alzheimer's disease.
